(19) United States
(12) Plant Patent Application Publication
(10) Pub. No.: US 2017/0347512 P1
(43) Pub. Date: Nov. 30, 2017

(54) Almond Variety Named 'CARINA'

(50) ;

(71) Applicants: ADELAIDE RESEARCH & INNOVATION PTY LTD., Adelaide (AU); HORTICULTURE INNOVATION AUSTRALIA LIMITED, Sydney (AU)

(72) Inventors: Michelle Wirthensohn, Adelaide (AU); Andrew Granger, Montacute (AU)

(21) Appl. No.: 14/999,573

(22) Filed: May 26, 2016

(51) Int. Cl. A01H 5/08

(52) U.S. Cl. CPC A01H 5/0837
USPC PLT/155

(57) ABSTRACT

A new and distinct almond variety of Prunus dulcis named ‘CARINA’, particularly characterized by self-fertility and very high production. Other desirable characteristics include very early to early harvest time, well-sealed semi-hard shells, and high quality, sweet kernels with high oil content.

Latin name of the genus and species of the plant claimed: Prunus dulcis.

Variety denomination: ‘CARINA’.

### BACKGROUND OF THE INVENTION

The present invention relates to a new and distinct cultivar of almond varieties, botanically known as Prunus dulcis, and hereinafter referred to by the name ‘CARINA’.

The disclosure provides a new and distinct variety of almond tree, botanically known as Prunus dulcis, synonymous with Prunus amygdalus Batsch., Amygdalus communis L., and Amygdalus dulcis Mill., which belongs to the Rosaceae family, and is hereinafter referred to by the variety denomination ‘CARINA’.

The new Prunus dulcis variety is a product of a controlled breeding program conducted by the inventors in Adelaide, Australia. The objective of the breeding program was to develop new almond varieties with high production, self-fertility and good kernel characteristics.

The new Prunus dulcis ‘CARINA’ originated from a cross in 1998 in Adelaide, Australia. The female or seed parent is Prunus dulcis variety designated ‘Nonpareil’ (unpatented) and the male or pollen parent is the French self-fertile Prunus dulcis variety designated ‘Lauranne’ (unpatented). The new Prunus dulcis ‘CARINA’ was selected by the inventors from the progeny of the stated cross in field trials in 2009 in Lindsay Point, Australia. First observations occurred in 2003.

Asexual propagation of the new Prunus dulcis ‘CARINA’ by grafting onto Prunus persica (L.) Batsch rootstock designated ‘Nemaguard’ (unpatented) was first performed in 2005 in the orchard located in Lindsay Point, Australia. Asexual propagation of the new Prunus dulcis ‘CARINA’ has confirmed that the characteristics as herein disclosed for the new variety are stable and retained through successive generations of asexual propagation. The new variety propagates true-to-type.

Asexual reproduction of the new almond tree has shown that the unique features of this new almond tree are stable and reproduced true to type in successive generations of asexual reproduction.

### SUMMARY OF THE INVENTION

The ‘CARINA’ variety of almond is of medium size, low to medium vigor with open growth and demonstrates very high and regular production of semi-hard nuts with kernels having an excellent flavour similar to ‘Nonpareil’ (unpatented). The harvest maturity is similar to ‘Nonpareil’ (unpatented) and the nuts release from the hulls readily. The percentage of doubles is low, usually less than 1% under growing conditions in the Riverland area of South Australia. The tree is self-fertile and, therefore, is able to produce almonds without the use of pollinators.

The following traits have been repeatedly observed and are determined to be the unique characteristics which make the new variety ‘CARINA’ clearly distinguishable from its parents and the variety most similar of common knowledge which is ‘Peerless’ (unpatented):

1. self-fertility;

2. high productivity; and

3. ease of harvest.

The following characteristics listed in Table 1 have been repeatedly observed in combination and distinguish ‘CARINA’ as a new and distinct almond variety:

TABLE 1

| | | | | Most similar |
|---|---|---|---|---|
| | | | | variety of |
| | | | | common |
| | | Female parent | Male parent | knowledge |
| | New variety | ‘Nonpareil’ | ‘Lauranne’ | ‘Peerless’ |
| Trait | ‘CARINA’ | (unpatented) | (unpatented) | (unpatented) |
| Shell type | Semi-hard | Paper | Hard | Semi-hard |
| Tree habit | Open | Slightly open | Spreading- | Open- |
| | | | drooping | spreading |
| Self-fertility | Present | Absent | Present | Absent |
| Flowering | Early | Early- | Late- | Early |
| time | | medium | very late | |
| Fruit size | Medium | Medium | Small | Medium- |
| | | | | large |
| Time of | Very early- | Early | Early- | Early- |
| maturity | early | | medium | medium |

Distinguishing characteristics of ‘CARINA’ are set out in Table 1. Plants of the new ‘CARINA’ almond tree have not been observed under all possible environmental conditions and cultural practices. The phenotype may vary somewhat with variations in environment, such as temperature, day length and light intensity, without, however, any variance in genotype.

The primary difference between the new variety and the female parent ‘Nonpareil’ (unpatented) is the new variety is self-fertile, whereas ‘Nonpareil’ (unpatented) is self-sterile and requires a pollinator tree planted near to fertilize the flowers and, thus, produce almonds. In comparison to its male parent ‘Lauranne’ (unpatented), the new variety blooms earlier by about 7-10 days, has larger fruit, and has an open tree habit.

The primary difference between the new variety and the most similar variety of common knowledge ‘Peerless’ (unpatented) is the new variety is self-fertile, whereas ‘Peerless’ (unpatented) is self-sterile and requires a pollinator tree planted near to fertilize the flowers and, thus, produce almonds.

### BRIEF DESCRIPTION OF THE DRAWING

The accompanying photographs (drawings) illustrate the overall appearance of the new Prunus dulcis ‘CARINA’ showing the colors, as true as is reasonably possible with digital reproduction. Colors in the photographs may differ slightly from the color values cited in the detailed botanical description, which accurately describe the color of ‘CARINA’. The trees were grown on Nemaguard rootstock.

FIG. 1 shows typical flowers of ‘CARINA’, dissected (FIG. 1A) and in situ (FIG. 1B).

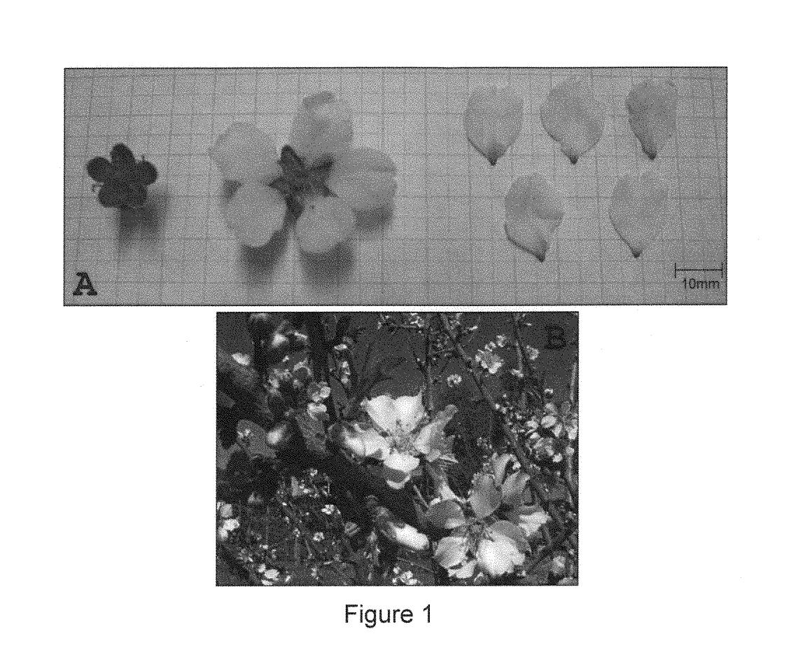

FIG. 2 shows various images of fruit of ‘CARINA’, including a one year-old shoot, showing green immature fruit (FIG. 2A), mature fruit in situ (FIG. 2B), and kernel and dry fruit shape (FIG. 2C).

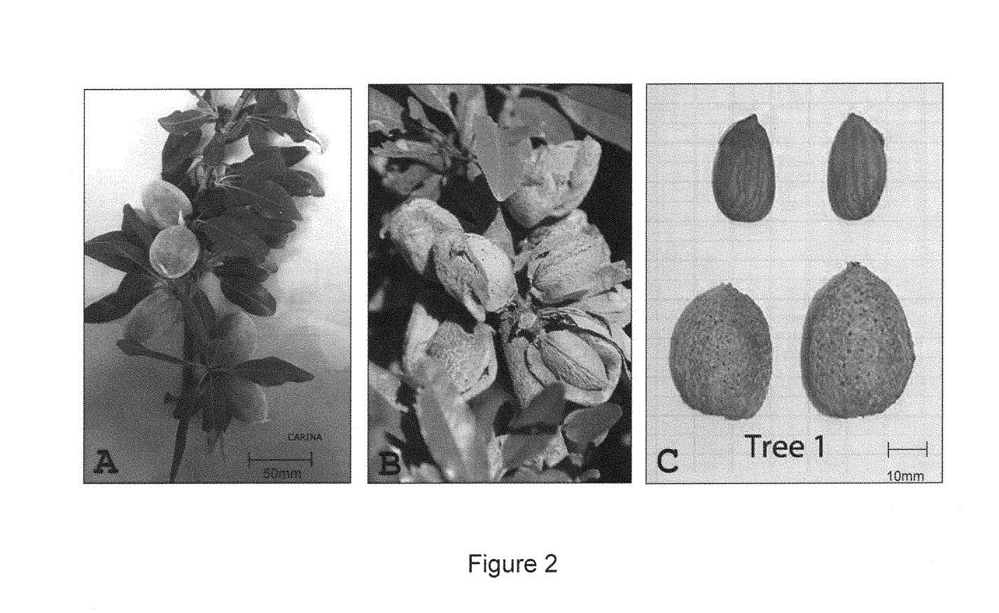

FIG. 3 shows a typical four year-old tree of ‘CARINA’ on 15 Sep. 2010.

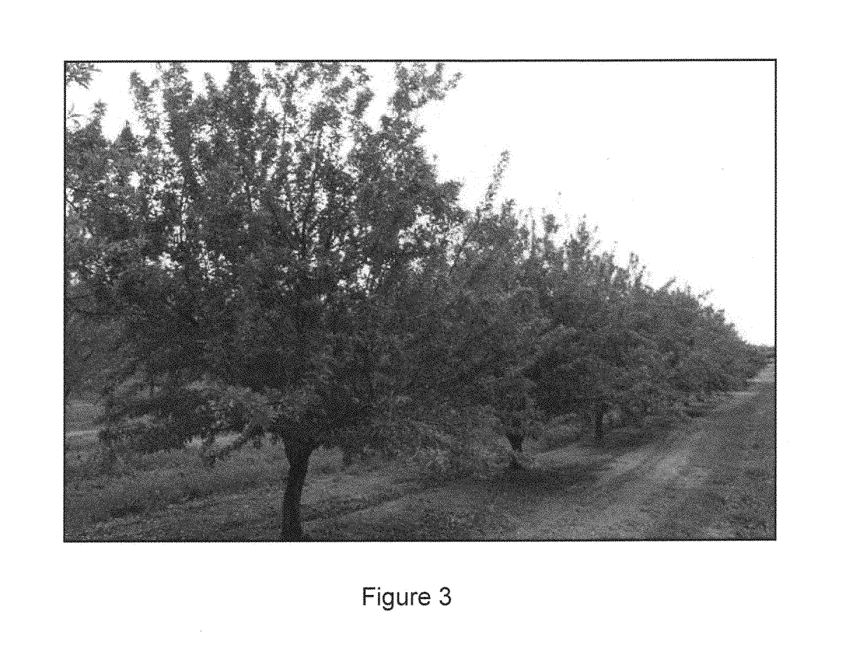

### DETAILED BOTANICAL DESCRIPTION

Plants used in the aforementioned photographs and in the following description were grown outside under natural season conditions and cultural practices which approximate those generally used in commercial almond production. During the production of the plants, day temperatures ranged from about 14.5° C. to 48.2° C., night ranged from about −5.7° C. to 14° C. and light levels ranged from about 126,905 to 564,729 foot-candles. Measurements and numerical values represent averages for typical flowering plants.

The following is a detailed description of the new ‘CARINA’ variety when observed during the growing seasons from 2010 to 2015 at Lindsay Point, Victoria, Australia. During 2015, the ‘CARINA’ trees were nine years of age. Quantified measurements are expressed as an average of measurements taken from a number of trees of ‘CARINA’. The measurements of any individual tree (or any group of trees) of ‘CARINA’ may vary from the stated average.

Color references are made to The Royal Horticultural Society Colour Chart (R.H.S.), sixth edition, (2015). Color values were taken under conditions of natural light.

All of the trees of ‘CARINA’, insofar as they have been observed, have been consistent in the characteristics described below.
Classification:
   Botanical.—Prunus dulcis.
Parentage:
   Female, or seed parent.—Prunus dulcis variety designated ‘Nonpareil’, unpatented.
   Male, or pollen parent.—French self-fertile Prunus dulcis variety designated ‘Lauranne’, unpatented.
Propagation:
   Type.—Budding onto rootstock.
   Time to initiate roots.—NA.
   Time to produce young plant.—Eight months.
   Root description.—‘Nemaguard’ (unpatented) rootstock.
Plant description:
Tree:
   Size.—Medium. Slightly smaller than Nonpareil.
   Vigor.—Low to medium.
   Density.—Medium to high.
   Habit.—Open with some limbs hanging over slightly.
Trunk:
   Texture.—Rough with some cracking.
   Color of bark.—RHS N200 B, brownish grey.
   Lenticels length.—2.3 mm.
   Lenticels width.—1.2 mm.
   Lenticels density.—10 per cm² on nine year-old wood.
   Lenticels shape.—Narrow elliptic.
   Lenticels color.—RHS 165A, moderate brown.
Current season shoot:
   Shape in cross section.—Round.
   Color.—RHS 143B, strong yellow green.
One year-old shoot:
   Thickness.—Thin to medium, 2.5-3.8 mm.
   Shape in cross section.—Round.
   Color.—RHS N199D, strong yellowish brown.
   Anthocyanin coloration.—Present.
   Intensity of anthocyanin coloration.—Strong.
   Feathering.—Very slight to slight.
   Lenticels.—Present.
Foliage:
   Density.—Medium.
Leaf blade:
   Length.—55-70 mm, average 62 mm.
   Width.—18-26 mm, average 23 mm.
   Length/width ratio.—Low.
   Shape.—Elliptic.
   Shape of base.—Obtuse.
   Shape of apex.—Obtuse.
   Color.—RHS NN137B, greyish olive green.
   Incisions of margin.—Crenate.
   Venation type.—Arcuate to pinnate.
Petiole:
   Length.—14-28 mm, average 20 mm.
   Color on upper side.—RHS 144A, strong yellow green.
   Shape in cross section.—Concave.
Flower buds:
   Distribution.—Intermediate.
   Shape.—Conical.
   Color of tip of petals.—RHS 62D, pale purplish pink.
   Color of sepals.—RHS 183B, dark red.
   Hairiness of sepals.—Medium.
Flower:
   Diameter.—38-45 mm, average 40 mm, medium.
   Shape of petals.—Elliptic.
   Shape of apex.—Retuse, indented.
   Shape of base.—Acute.
   Number of petals.—Five.
   Color of petals.—RHS N155D, yellowish white.
   Number of stamens.—Medium to many.
   Number of pistils.—Always one.
   Position of stigma as compared with anthers.—Below.
Stamen:
   Anthocyanin coloration of filament.—Present.
Stigma:
   Size.—Large.
Green fruit:
   Size.—Large.
   Shape.—Ovate.
   Pubescence.—Much.
Dry fruit:
   Shape.—Ovate.
   Shape of apex.—Pointed.
   Length.—26-34 mm.
   Width.—18-25 mm.
   Thickness.—14-17 mm.
   Average weight.—2.7 g.
   Color.—RHS 164B, moderate orange yellow.
   Surface.—Smooth with moderate pitting.
   Thickness of endocarp.—2-3 mm.
   Resistance to cracking.—Medium, semi-hard.
   Percentage of kernel to dry fruit.—Approximately 34%.
   Keel development.—Weak.
   Distribution on tree.—On spurs and one year-old shoots.
Fruit:
   Percentage of double kernels.—Low, less than 1%.
   Eating quality.—Excellent. Oil content average is 57.4%; Oleic acid 62.3%, Vitamin E 53.8 mg/100 g oil.
Kernel:
   Shape.—Broad elliptic.
   Size.—Small.
   Average weight.—1 g.
   Length.—19-24 mm.
   Width.—11-15 mm.
   Thickness.—7-9 mm.
   Main color.—RHS 164B, moderate orange yellow.
   Intensity of color.—Light.
   Rugosity.—Weak.
   Taste.—Sweet.
Blooming/flowering timing:
   Time of beginning of flowering.—Early, up to five days before ‘Nonpareil’.
   Time of leaf budburst in relation to beginning of flowering.—Simultaneous to later.
   Flowering period.—Up to 29 days depending on the weather.
   Time of maturity.—Very early to early.
Cultural characteristics:
   Susceptibility to disease.—Very good tolerance to bacterial spot.

We claim:

1. A new and distinct variety of almond tree (Prunus dulcis) named ‘CARINA’, as illustrated and described herein.
